(19) United States
(12) Patent Application Publication
(10) Pub. No.: US 2019/0167649 A1
(43) Pub. Date: Jun. 6, 2019

(54) TOPICAL RAPAMYCIN THERAPY

(71) Applicant: THE BOARD OF REGENTS OF THE UNIVERSITY OF TEXAS SYSTEM, Austin, TX (US)

(72) Inventors: Mary Kay KOENIG, Houston, TX (US); Hope NORTHRUP, Houston, TX (US); Adelaide A. HEBERT, Houston, TX (US)

(21) Appl. No.: 16/324,274

(22) PCT Filed: Aug. 10, 2017
(86) PCT No.: PCT/US2017/046320

§ 371 Date: Feb. 8, 2019

Related U.S. Application Data
(60) Provisional application No. 62/372,940, filed on Aug. 10, 2016.

(51) Int. Cl. A61K 31/436; A61K 47/10; A61K 9/06; A61K 9/00; A61P 17/02

(52) U.S. Cl. CPC A61K 31/436; A61K 47/10; A61P 17/02; A61K 9/0014; A61K 9/06

(57) ABSTRACT

A topical composition containing rapamycin and an alcohol-free carrier is used to deliver rapamycin to skin cells while preventing systemic absorption, allowing for safe and effective treatment of various skin conditions, including angiofibromas that arise from conditions such as Tuberous Sclerosis Complex (TSC).

This application claims the benefit of U.S. Provisional Patent Application No. 62/372,940, filed Aug. 10, 2016, the entirety of which is incorporated herein by reference.

This invention was made with government support under Grant No. W81XWH-11-1-0240 awarded by the Department of Defense. The government has certain rights in the invention.

### BACKGROUND

This disclosure pertains to a topical rapamycin therapy for use in the treatment of skin conditions.

The skin is a complex organ covering the external surfaces of the body. Skin conditions, particularly conditions affecting highly visible portions of the skin such as the face and arms, have a tremendous psychosocial impact on an individual, his or her family, and their quality of life. The human face is considered perhaps the most important element of the social environment. A number of significant, debilitating skin disorders persist which impact individuals daily on a personal and social level.

One such disorder is Tuberous Sclerosis Complex (TSC), a genetic disorder characterized by alterations in skin pigmentation and tumor formation in multiple organ systems, including the skin. TSC results from mutations in either the TSC1 gene (hamartin) or the TSC2 gene (tuberin). Although TSC is inherited in an autosomal dominant fashion, there is considerable variability in presentations and symptoms range from mild to severe. Common symptoms of TSC include learning disabilities/mental retardation, seizures, skin lesions, kidney tumors, lung disease, heart tumors, and brain tumors. Cells with non-functional TSC genes secrete vascular growth factors that induce angiogenesis. The overproduction of skin cells, in conjunction with angiogenesis, results in the formation of visible facial angiofibromas over time. The angiofibromatous lesions appear as flesh colored to red or pink papules distributed over the central face, especially on the nasolabial folds, cheeks, and chin. Lesions appear in early childhood and are present in up to 80% of TSC patients.

Facial angiofibromas create considerable cosmetic morbidity for patients with TSC. Since initial descriptions in the 19^{th} Century, multiple treatments have been developed to alleviate the appearance of these lesions (curettage, cryosurgery, chemical peels, dermabrasion, shave excisions, and laser therapy). Although the majority of these treatments are effective, they are uncomfortable and often need to be repeated at periodic intervals to treat recurrence. Currently there is no effective method for preventing or permanently removing facial angiofibromas in patients with TSC.

Rapamycin is also referred to by its generic drug name, sirolimus (see for example, ANDA #201578, by Dr. Reddys Labs Ltd., approved May 28, 2013). Sirolimus is FDA approved and marketed in the United States for the prophylaxis of organ rejection and renal transplantation under the trade name RAPAMUNE by Wyeth (Pfizer). RAPAMUNE is available in the form of an oral solution (1 mg/ml) or tablet (multiple strengths). Wyeth (Pfizer) also markets a derivative by the tradename TORISEL (temsirolimus) for the treatment of advanced renal cell carcinoma, which is administered intravenously. Temsirolimus is a water-soluble prodrug of sirolimus. Cordis, a division of Johnson & Johnson, markets a sirolimus-eluting coronary stent under the tradename CYPHER. In this context, the antiproliferative effects of sirolimus prevent restenosis in coronary arteries following balloon angioplasty. US 2010/0305150 to Berg et al. (Novartis) describes rapamycin derivatives for treating and preventing neurocutaneous disorders, such as those mediated by TSC including tuberous sclerosis, as well as those mediated by neurofibromatosis type 1 (NF-1). Rapamycin and its derivatives are further described in Nishimura, T. et al. (2001) Am. J. Respir. Crit. Care Med. 163:498-502 and in U.S. Pat. Nos. 6,384,046 and 6,258,823.

U.S. Pat. No. 7,416,724 (Regents of the University of Michigan) describes methods of treating a subject with tuberous sclerosis comprising administering to said subject an effective amount of rapamycin. US 2013/0225630 (Innova Dermaceuticals) describes methods of treating facial angiofibromas by applying an effective amount of topical rapamycin, which is described in the specification as from about 0.25% to about 2% by weight. U.S. Pat. No. 6,958,153 (Wyeth) describes a topical formulation for the treatment of “a dermatological condition” which comprises rapamycin and a permeation modulator present in relative amounts such that when a therapeutic amount is applied to the skin a minimal systemic effect is produced.

A review published in 2015 analyzed the current data on the use of topical rapamycin in treating facial angiofibromas in TSC. Balestri et al., J. Eur. Acad. Derm. Venereology (2015), 29(1), 14-20. Sixteen reports involving a total of 84 patients were considered, and among these, an improvement of the lesions was reported 94% of patients. These study notes that several different formulations, e.g., ointments, gels, solutions, and creams were used, over a range of rapamycin concentrations from 0.003% to 1%.

One previous trial investigated the safety and efficacy of low dose (0.003% or 0.015%) topical rapamycin for treating facial angiofibromas in patients with TSC. Koenig et al., Drugs in R&D (2012), 12(3), 121-126. This was a small study (23 subjects) in which efficacy was assessed based on a subjective measure, namely the patient's self-reported assessment of whether or not treatment improved their condition, made it worse, or had no effect. Using this measure, less than (but almost) half of the patients in the combined treatment arms reported improvement. The authors note that the results did not reach statistical significance, meaning that a treatment effect of the vehicle alone could not be ruled out.

Although considerable effort has been made to develop an effective topical rapamycin formulation for treating facial angiofibromas, to date there remains a need for a safe and effective formulation. The present disclosure addresses that need.

### SUMMARY

The present disclosure relates to topical rapamycin compositions and their use in methods for treating facial angiofibromas, and other skin lesions associated with aberrant activation of mTOR signaling.

The disclosure provides compositions for use in treating facial angiofibromas, or other skin lesions, in a patient in need thereof, the compositions comprising rapamycin in an amount of from 0.1% to 5% by weight, a liquid glycol, and a dermatologically acceptable carrier, wherein the composition is free of added alcohols, for example, ethyl alcohol, isopropyl alcohol, and specially denatured (SD) alcohol.

In embodiments, the patient in need of treatment is a patient diagnosed with Tuberous Sclerosis Complex (TSC).

In embodiments, the other skin lesions are selected from hemangiomas, vascular malformations, pyogenic granulomas, essential telangiectasias, familial multiple discoid fibromas, and cherry angiomas. In embodiments, the vascular malformations are port wine stains or lymphangiomas. In embodiments where the use is for treating other skin lesions, the patient in need of treatment is a patient diagnosed with Proteus, Brooke-Speigler syndrome, nevus sebaceous, epidermal nevus, oral lichen planus, chelitis granulomatosis, neurofibromatosis type 1, overgrowth syndromes, or gingival hypertrophy.

In embodiments, the rapamycin is present in an amount of about 0.1% to about 1%, about 0.5% to 1%, about 1% to 3%, 2.5% to 5%, or from about 3% to 5% by weight based on the total weight of the composition. In embodiments, the rapamycin is present in an amount of 1%, 2%, 3%, 4%, or 5% by weight based on the total weight of the composition.

In embodiments, the composition is free of added formaldehyde, fragrances, and dyes. In embodiments, the composition is free of added acetone. In embodiments, the composition is free of added polyvinylidene fluoride.

In embodiments, the liquid glycol is selected from propylene glycol, polyethylene glycol, and glycerol. In embodiments, the liquid glycol is propylene glycol.

In embodiments, the carrier comprises one or more of purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT).

In embodiments, the disclosure provides a composition for use in treating facial angiofibromas in a patient in need thereof, the composition comprising rapamycin in an amount of greater than 2% up to 5%, or from 3% to 5% by weight, a liquid glycol, preferably selected from propylene glycol, polyethylene glycol, and glycerol, and most preferably propylene glycol, in an amount of less than 10%, preferably from 1-8% by weight, and a dermatologically acceptable carrier, wherein the composition is free of added alcohol, for example, ethyl alcohol, isopropyl alcohol, and specially denatured (SD) alcohol, and free of added acetone, and polyvinylidene fluoride, and all weight percentages are based on the total weight of the composition.

In embodiments, a composition described here is effective to treat facial angiofibromas as determined by a reduction in average size and number of lesions.

In embodiments, the disclosure provides a method for reducing the appearance of skin lesions on an affected area of the skin of a patient, comprising applying to the affected area a topical rapamycin-containing composition, wherein the topical rapamycin-containing composition comprises rapamycin in an amount of about 0.1% to about 5% by weight and an alcohol-free carrier, wherein the rapamycin penetrates into the skin of the patient, and wherein the rapamycin is not systemically absorbed by the patient. In embodiments, the method further comprises observing a reduction in the appearance of the skin lesions on the affected areas of the skin of the patient. In embodiments, the method further comprises a step of re-applying the topical rapamycin-containing composition on the affected areas of the skin of the patient a desired number of times. In embodiments, the skin lesions are angiofibromas resulting from Tuberous Sclerosis Complex (TSC). In other embodiments, the skin lesions are hemangiomas, vascular malformations, pyogenic granulomas, essential telangiectasias, familial multiple discoid fibromas, or cherry angiomas. In an embodiment, the vascular malformations are port wine stains or lymphangiomas. In embodiments, the skin lesions result from Proteus, Brooke-Speigler syndrome, nevus sebaceous, epidermal nevus, oral lichen planus, chelitis granulomatosis, neurofibromatosis type 1, overgrowth syndromes, or gingival hypertrophy.

In embodiments of the compositions and methods described here, the rapamycin is present in an amount of about 0.1% to about 1% by weight, based on the total weight of the composition.

In embodiments of the compositions and methods described here, the carrier is free of added acetone, formaldehyde, fragrances, and dyes. In embodiments, the carrier comprises purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT). In embodiments, the topical rapamycin-containing composition further comprises one or more sunscreens. In embodiments, the topical rapamycin-containing composition further comprises one or more cosmetic ingredients. In embodiments, the topical rapamycin-containing composition further comprises one or more formulation materials.

### BRIEF DESCRIPTION OF THE DRAWINGS

FIG. 1 shows a diagram of a scoring chart used for calculating an Angiofibroma rating scale (AGS) in evaluating the appearance of angiofibromas in patients treated with embodiments of the topical rapamycin composition in accordance with the present disclosure.

FIG. 2 shows change in average AGS for three treatment arms (placebo, low, and high) in a trial evaluating embodiments of the topical rapamycin composition, as reported for two dermatologists (indicated by shorthand as “s” and “y”).

FIG. 3 shows change in overall AGS for the three treatment arms (placebo, low, and high) in a trial evaluating embodiments of the topical rapamycin composition, combined for both dermatologists.

FIG. 4A shows the proportion of patients in each treatment arm having less prominent lesions after treatment compared to baseline, as reported by a first dermatologist.

FIG. 4B shows the proportion of patients in each treatment arm having less prominent lesions after treatment compared to baseline, as reported by a second dermatologist.

FIG. 4C shows the average of the reports by the two dermatologists for the proportion of patients in each treatment arm having less prominent or the same prominence of lesions after treatment compared to baseline.

FIG. 4D shows the average of the reports by the two dermatologists for the proportion of patients in each treatment arm having less prominent lesions after treatment compared to baseline.

FIG. 4E shows the average of the reports by the two dermatologists for the proportion of patients in each treatment arm having more prominent lesions after treatment compared to baseline.

FIG. 5 shows the proportion of patients in each treatment arm who reported improved Dermatology Quality of Life Index (DQLI) between visits 1 and 7 in a trial evaluating embodiments of the topical rapamycin composition.

FIG. 6 shows the change in DQLI for patients in each treatment arm between visits 7 and 1 in a trial evaluating embodiments of the topical rapamycin composition.

FIG. 7 shows the change in DQLI from baseline for patients in each treatment arm for baseline and visits 4 and 7 in a trial evaluating embodiments of the topical rapamycin composition.

### DETAILED DESCRIPTION OF PREFERRED EMBODIMENTS

Generally, the present disclosure relates to topical rapamycin compositions that are alcohol-free and effective to deliver a therapeutically effective amount of rapamycin to skin cells without appreciable systemic absorption of the rapamycin, allowing for safe and effective treatment of skin conditions amendable to topical treatment with an inhibitor of the mTOR pathway, also referred to herein as topical mTOR-related diseases and disorders. In embodiments, the topical mTOR-related disease or disorder is Tuberous Sclerosis Complex (TSC).

In the present context, “without appreciable systemic absorption of rapamycin” means blood levels of rapamycin that are less than 2 ng/ml, or preferably less than 1 ng/ml, within 12 or 24 hours after application to the skin.

In embodiments of the compositions and methods described here, the patient in need of treatment is a human subject diagnosed with TSC. In the context of the present disclosure, the term “patient” generally refers to a human subject having a diagnosis.

TSC is a genetic disorder relating to mutations in the TSC1 and TSC2 genes. These genes are involved in the mammalian target of rapamycin (mTOR) signaling pathway. mTOR is found in the cytoplasm complexed with several other molecules. The main function of mTOR is to stimulate protein synthesis, cell survival, and cell cycle progression. In a nutrient poor state, TSC1 and TSC2 gene products form a complex inside the cell cytoplasm (the TSC1-TSC2 complex). In this active (complexed) state, the TSC1-TSC2 complex inhibits mTOR. mTOR inhibition prevents cell growth, protein synthesis and cell division. In the nutrient rich state, nutrients (amino acids, glucose, and oxygen) enter the cytoplasm by passive diffusion and, through a series of steps, phosphorylate the TSC2 gene product. Once phosphorylated, the TSC2 gene product dissociates from the TSC1 gene product, thus relieving inhibition of mTOR activity and allowing cell growth, protein synthesis, and cell division. In the TSC disease state, either the TSC1 or TSC2 gene product is defective, resulting in an inability of the gene products to complex, thus preventing the inhibition of mTOR signaling. Cells thus live in a continuous state of uninhibited mTOR activity, resulting in cell overgrowth and tumor formation. In patients with TSC, it is the epidermal basal cells of the skin that contain a mutant copy of either the TSC1 or TSC2 gene. A loss of heterozygosity results in a constitutive activation of mTOR with subsequent production of epidermal cells at a faster rate than the ability to slough off dead cells from the superficial stratum corneum. Cells with non-functional TSC genes also secrete vascular growth factors. Rapamycin binds with high specificity to mTOR resulting in inhibition of the activity of mTOR and ultimately in downregulation of cell growth.

The present topical rapamycin therapy limits systemic absorption of rapamycin to very low or undetectable levels (less than 2 ng/ml, or preferably less than 1 ng/ml blood levels), thereby providing a safe, effective treatment for facial angiofibromas in patients with TSC, as well as other skin lesions characterized by aberrant activation of the mTOR signaling pathway.

In embodiments, the topical composition is formulated with a cream base. In the context of the present disclosure, the terms ‘base’ and ‘carrier’ are used interchangeably. The term base has its ordinary meaning as it is used in the context of the science of pharmaceutics. Briefly, a cream base is a stable emulsion of oil and water. In some aspects, a cream base comprises oil and water in approximately equal proportions. Other optional excipients may be included in the base, as described in more detail infra. For example, the base may further comprise an antimicrobial agent, a buffering agent, a surfactant, etc.

Embodiments of the topical rapamycin compositions described here include rapamycin in amounts of about 0.1% to about 5% by weight, or preferably about 0.1% to about 1% by weight, in a non-comedogenic, moisturizing carrier that lacks alcohol, for example, ethyl alcohol, isopropyl alcohol, and specially denatured (SD) alcohol, and other drying agents or irritants.

In embodiments, the compositions contain rapamycin in relatively high amounts, for example, in amounts of greater than 1%, or greater than 2%, and up to about 5% by weight, based on the total weight of the composition.

In embodiments, the present disclosure provides a topical composition containing rapamycin as the active pharmaceutical ingredient (“API”). In embodiments, rapamycin is the only API in the composition. Rapamycin is also referred to as sirolimus, and in the context of the present disclosure the terms “rapamycin” and “sirolimus” may be used interchangeably.

Rapamycin is a macrocyclic lactone produced by Streptomyces hygroscopicus. Its molecular formula is C₅₁H₇₉NO₁₃ and its molecular weight is 914.172 g/mol, allowing for its absorption through the superficial layers of the human epidermis. In accordance with one aspect of the present invention, an appropriate delivery system allows topically applied rapamycin to penetrate the skin and reach the deep epidermal basal cells implicated in development of facial angiofibromas.

Isomers of rapamycin are known, e.g., isomer B and isomer C, having structures as shown in U.S. Pat. No. 7,384,953. Typically, rapamycin is a mixture of the B and C isomers. In solution, rapamycin isomers B and C interconvert and an equilibrium is achieved. In embodiments of the compositions and methods described here, the API is rapamycin having an isomeric B:C ratio of greater than 30:1 or greater than 35:1. In one embodiment, the rapamycin has an isomeric B:C ratio of 3.5% to 10%. It is common practice in the literature to depict the structure of rapamycin in the form of the B isomer, which is the form shown below.

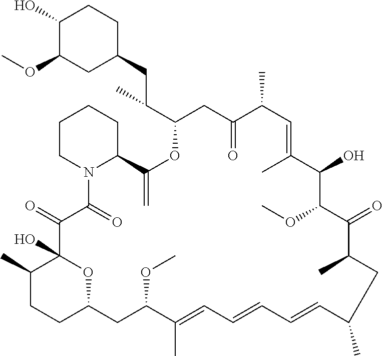 [SMILES: C=C1O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]2CC[C@@H](C)[C@@](O)(O2)C(=O)C(=O)N2CCCC[C@@H]12]

Rapamycin is a white to off-white powder and is considered insoluble in water, having a very low solubility of only 2.6 μg/ml. Rapamycin is freely soluble in benzyl alcohol, chloroform, acetone, and acetonitrile. In accordance with the present disclosure, it is preferred that the topical rapamycin composition does not contain added benzyl alcohol, chloroform, acetone, or acetonitrile.

In embodiments of the compositions and methods described here, the present disclosure provides a topical composition containing a rapamycin derivative, metabolite, or analog as the active pharmaceutical ingredient (“API”). In embodiments, the rapamycin derivative, metabolite, or analog is the only API in the composition. In embodiments, the API is a rapamycin derivative, metabolite, or analog selected from the group consisting of everolimus (Affinitor; RAD001), temsirolimus (CCI-779), ridaforolimus (previously known as deforolimus; AP23573), umirolimus (Biolimus A9), zotarolimus (ABT-578), novolimus, and myolimus. In embodiments, the API is a rapamycin derivative or analog selected from everolimus, ridaforolimus, umirolimus, and zotarolimus. Further derivatives are known to the skilled person and include, for example, an O-substituted derivative in which the hydroxyl group on the cyclohexyl ring of sirolimus is replaced by —OR₁, in which R₁ is optionally a substituted alkyl, acylaminoalkyl, or aminoalkyl.

The results presented infra indicate that mTOR inhibitors generally may be useful in treating facial angiofibromas and other skin conditions characterized by aberrant activation of mTOR signaling. Accordingly, in embodiments of the compositions and methods described here, the API is an mTOR inhibitor selected from AP23841, KU-0063794, INK-128, EX2044, EX3855, EX7518, AZD08055 and OSI027. In embodiments, the mTOR inhibitor selected from the group consisting of KU-0063794, AZD8055, INK128, and OSI-027.

In embodiments, the present disclosure provides a topical rapamycin composition that includes rapamycin, propylene glycol, and a carrier. In embodiments, the carrier may be a commercially available carrier such as Vanicream™ skin cream (Pharmaceutical Specialties, Inc., Rochester, Minn.). This exemplary carrier is made up of purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT). This carrier is non-greasy, moisturizing, easily spreadable, quickly absorbed, and non-comedogenic.

Preferably, the topical rapamycin composition does not contain added alcohol, for example, ethyl alcohol, isopropyl alcohol, and specially denatured (SD) alcohol, or added acetone, or formaldehyde. In embodiments, the composition further lacks other irritating ingredients such as dyes and fragrances. In embodiments, the composition does not contain added polyvinylidene fluoride.

In embodiments, the topical rapamycin composition contains rapamycin in an amount of about 0.1% by weight, up to about 5% by weight, based on the total weight of the composition. In embodiments, the topical rapamycin composition contains rapamycin in an amount of greater than 2%, up to about 5% by weight, based on the total weight of the composition. In embodiments, the topical rapamycin composition contains rapamycin in an amount of about 0.1% by weight, up to about 1% by weight, based on the total weight of the composition. In certain embodiments, the amount of rapamycin may be 0.5%, 1%, 2%, 3%, 4%, or 5% by weight, based on the total weight of the composition.

All amounts of the ingredients in the compositions described here are weight percentages (wt %) based on the total weight of the composition, unless explicitly noted otherwise.

The disclosure provides methods of treating facial angiofibromas, or other skin lesions, in a patient in need of such treatment, by applying to the affected area of skin a topical composition as described herein. As discussed above, a patient in need of treatment is one who is diagnosed, for example, with a disease or disorder characterized by facial angiofibromas or other skin lesions, such as hemangiomas, vascular malformations, pyogenic granulomas, essential telangiectasias, familial multiple discoid fibromas, and cherry angiomas. In embodiments, the patient is one who is diagnosed with TSC. In other embodiments, the patient is one who is diagnosed with Proteus, Brooke-Speigler syndrome, nevus sebaceous, epidermal nevus, oral lichen planus, chelitis granulomatosis, neurofibromatosis type 1, overgrowth syndromes, or gingival hypertrophy.

In the context of the methods described here, the terms “treat”, “treatment”, and “treating” refer to the reduction of the severity, duration, or progression of the skin lesions, for example as assessed by clinical parameters including one or more of the presence and/or degree of erythema, the average lesion size, the density of the lesions in an affected area, and the percent involvement. In embodiments, “treating” may also encompass reducing the appearance of new skin lesions, such as facial angiofibromas, hemangiomas, vascular malformations, pyogenic granulomas, essential telangiectasias, familial multiple discoid fibromas, and cherry angiomas.

In embodiments, the amount of rapamycin in a composition described here is an amount effective to treat facial angiofibromas, or other skin lesions, including hemangiomas, vascular malformations, pyogenic granulomas, essential telangiectasias, familial multiple discoid fibromas, and cherry angiomas.

In embodiments, the effective amount of rapamycin is the amount applied to the skin according to the methods described here for application of a topical rapamycin composition. For example, in accordance with the methods described here, the amount of the composition applied to the affected area is generally in the range of about 5 cubic centimeters (cm³), or from about 5-20 cm³, or about 15-20 cm³. In accordance with the methods described here, the composition is applied to the affected area of skin, which is the area of skin comprising the lesions to be treated, in an amount suitable to cover the affected area with a thin layer of the composition, for example an amount in the range of about 5-20 cm³, or from about 15-20 cm³ applied to the affected area, preferably once daily or twice daily. In embodiments, the application is once daily. In embodiments, the topical rapamycin composition is applied topically to affected regions, such as the face, of a patient. In embodiments, a pump is used to dispense a defined amount of the composition, for example about 1 gm (or about 5-20 cm³ or from about 15-20 cm³). The dispensed amount is applied to affected regions of the skin and allowed to remain, preferably overnight, without wetting or washing. The composition is preferably stored and used at room temperature.

In embodiments, the methods comprise applying to the affected areas of the skin of the patient a topical rapamycin composition as described herein, wherein the composition comprises rapamycin in an amount of from 0.1% to 5% by weight, or from 0.1% to about 1%, about 0.5% to 1%, about 1% to 3%, 2.5% to 5%, or from about 3% to 5% by weight, based on the total weight of the composition. In embodiments, the amount of rapamycin is more than 2%, up to 5% by weight, based on the total weight of the composition.

As discussed above, the affected areas of the skin may contain facial angiofibromas. Alternatively, the affected areas may contain another type of skin lesion, such as hemangiomas, pyogenic granulomas, essential telangiectasias, vascular malformations such as port wine stains, lymphangiomas, familial multiple discoid fibromas, or cherry angiomas.

In additional embodiments of the methods described here, the rapamycin is present in an amount of about 0.1% to about 5%, an amount of about 0.5% to about 3%, an amount of about 1.0% to about 3%, or an amount of about 0.1% to about 1%. The method may also include the step of re-applying the topical rapamycin-containing composition on the affected areas of the skin of the patient a desired number of times. In preferred embodiments, the carrier is free of acetone, formaldehyde, fragrances, and dyes. In embodiments, the carrier comprises purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT).

In an exemplary embodiment, a topical rapamycin composition is prepared by mixing 1 gm of rapamycin powder with 2 ml propylene glycol to make a paste. About 10 gm of a suitable carrier (such as Vanicream™) is added to the paste and mixed well. Additional carrier is added to make the total mixture exactly 100 gm. The mixture is placed in a mixer or compounder such as an Unguator® (GAKO® International GmbH, Munich, Germany) and mixed for five minutes to produce a 1% rapamycin cream.

In additional embodiments, a topical rapamycin composition is prepared by mixing 10 gm of the 1% rapamycin cream described above with 90 grams of additional carrier, to make the total mixture exactly 100 gm. The mixture is placed in a mixer or compounder such as an Unguator® (GAKO® International GmbH, Munich, Germany) and mixed for five minutes to produce a 0.1% rapamycin cream.

As discussed in the example below, the results of a clinical study showed no detectable systemic absorption from topical compositions containing up to 1% by weight rapamycin. It is expected that the amount of rapamycin can be increased up to about 5% and maintain low (less than 2 ng/ml blood levels) or undetectable (less than 1 ng/ml blood levels) of rapamycin.

In addition, although the example pertains to treatment of angiofibromas arising in TSC patients, the efficacy of the topical rapamycin compositions described here in this indication suggests that their use can be extended to the treatment of other skin lesions, particularly those associated with aberrant mTOR activity. Accordingly, the present disclosure also provides methods of treating other types of skin lesions including, but not limited to, hemangiomas, vascular malformations, pyogenic granulomas, essential telangiectasias, port wine stains, lymphangiomas, proteus, familial multiple discoid fibromas, Brooke-Speigler syndrome, nevus sebaceous, epidermal nevus, cherry angiomas, oral lichen planus, chelitis granulomatosis, gingival hypertrophy (primary and secondary), overgrowth syndromes, and neurofibromatosis type 1 (NF-1) associated skin lesions.

As discussed above, in formulating the topical compositions described here, it is preferable to utilize a carrier that is alcohol-free and preferably free of other potential irritants such as acetone, formaldehyde, fragrances, and dyes. It is also preferred to utilize a carrier that effectively solubilizes rapamycin powder and maintains its stability. Other characteristics of the carrier may include its ease of spreadability onto skin, comfortable feel after application to skin, and lack of irritation to skin. In embodiments, the carrier comprises one or more of purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT), but these precise ingredients are not all strictly necessary, so long as the resulting carrier has the properties that are desired. The white petrolatum is non-irritating and is believed to hold, and possibly concentrate, the rapamycin at the skin surface where the medication can favorably impact the overgrowth that results in the angiofibroma formation and proliferation. The simethicone is soothing to the skin surface as well.

In some embodiments the topical rapamycin compositions further comprise one or more additives. In embodiments, the one or more additives may comprise a sunscreen or sun block, such as, but are not limited to, p-Aminobenzoic acid (PABA: (0-15%), Padimate O (OD-PABA, octyldimethyl-PABA, σ-PABA: 0-10%), Phenylbenzimidazole sulfonic acid (Ensulizole, Eusolex® 232 (Merck, Kenilworth, N.J.), PBSA, Parsol® HS (DSM Nutritional Products, Basel): 0 to 8%), Cinoxate (2-Ethoxyethyl p-methoxycinnamate: 0-6%), Dioxybenzone (Benzophenone-8: 0-3%), Oxybenzone (Benzophenone-3, Eusolex® 4360, Escalol™567 (Ashland, Garland, Tex.): 0-10%), Homosalate (Homomethyl salicylate, HMS: 0-15%), Menthyl anthranilate (Meradimate: 0-5%), Octocrylene (Eusolex® OCR, 2-Cyano-3,3-diphenyl acrylic acid, 2-ethylhexylester: 0-10%), Octyl methoxycinnamate (Octinoxate, EMC, OMC, Ethylhexyl methoxycinnamate, Escalol 557, 2-Ethylhexyl-paramethoxycinnamate, Parsol MCX: 0-20%) Octyl salicylate (Octisalate, 2-Ethylhexyl salicylate, Escalol™ 587: 0-10%), Sulisobenzone (2-Hydroxy-4-Methoxybenzophenone-5-sulfonic acid, 3-Benzoyl-4-hydroxy-6-methoxybenzenesulfonic acid, Benzophenone-4, Escalol™ 577: 0-10%), Trolamine salicylate (Triethanolamine salicylate: 0-12%), Avobenzone (1-(4-methoxyphenyl)-3-(4-tert-butyl phenyl)propane-1,3-dione), Butyl methoxy dibenzoylmethane (BMDBM, Parsol® 1789, Eusolex® 9020: 0-10%), Ecamsule (Mexoryl SX, Terephthalylidene Dicamphor Sulfonic Acid: 0-10%) and in preferred embodiments, physical sun blocks such as, but not limited to, Titanium dioxide (0-25% or more) and Zinc oxide (0-25% or more).

In embodiments, the one or more additives may comprise one or more cosmetic ingredients, such as a foundation or concealer, or appropriate ingredients for producing a desired shade or color. These cosmetic ingredients may include one or more pigments such as titanium dioxide, iron oxide, zinc oxide, kaolin, or variations or combinations thereof. For example, rutile or anatase titanium dioxide may be included, as well as red, yellow and black iron oxides. Cosmetic ingredients such as these typically require emulsion stability in conjunction with the proper color, and color maintenance over time, throughout the product. This typically requires a balance between oil and water phase interactions, emulsifiers, film-formers and different powders and pigments. The emulsions that are most often used to formulate foundations can be o/w and w/o, and silicones are typically used. A preferred example of a cosmetic foundation may include a combination of pigments including titanium dioxide (about 15 wt %), yellow iron oxide (about 2.85 wt %), red iron oxide (about 1.35 wt %), and black iron oxide (about 1.1 wt %). This combination of cosmetic ingredients may be used in the topical rapamycin-containing composition as well, with the appropriate weight percentages adjusted as desired. Additional ingredients used with the pigments may include fillers such as talc, mica, and methicone, skin adhesion ingredients such as zinc stearate, preservatives such as methylparaben or propylparaben, and binders such as coco caprylate/caprate. These may be additionally used in the topical rapamycin-containing composition in conjunction with the cosmetic ingredients.

In embodiments, the one or more additives may be selected from antimicrobials; antioxidants (for example, ascorbic acid, sodium sulfite and sodium hydrogen-sulfite); buffers (for example, borate, bicarbonate, Tris-HCl, citrates, phosphates and other organic acids); chelating agents (for example, ethylenediamine tetraacetic acid (EDTA)); complexing agents (for example, polyvinylpyrrolidone, beta-cyclodextrin, and hydroxypropyl-beta-cyclodextrin); emulsifying agents; hydrophilic polymers (for example, polyvinylpyrrolidone); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents (for example, nanoparticles); Poloxamer 188; Poly(d,-lactic acid); Propylene carbonate; surfactants or wetting agents (for example, pluronics, PEG, sorbitan esters, polysorbates (for example, polysorbate 20 and polysorbate 80), Triton™ (Dow Chemical, Midland, Mich.), tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and other recognized pharmaceutical adjuvants. “Remington: The Science and Practice of Pharmacy”, 20th edition (Gennaro, LWW, Dec. 15, 2000), 22nd edition, (Allen, Loyd V., Pharmaceutical Press, Sep. 15, 2012).

In embodiments, the one or more additives includes niacinamide.

### Example: Clinical Study

A study was conducted to evaluate topical rapamycin for reducing angiofibroma size and suppressing new angiofibroma growth resulting from TSC. Ten clinical study sites were utilized—nine in the U.S. and one in Australia. A total of 177 individuals enrolled in the study and were assigned to three treatment arms: placebo (n=58), low dose rapamycin, 0.1% (n=61), and high dose rapamycin, 1.0% (n=58). 154 individuals completed the trial.

To ensure equal subjects in each arm at each site, computerized block randomization was performed, stratified at each site. A block size of 6 was used (2 of each treatment). Throughout the trial, treatment allocation was masked to subjects, personnel, and outcome assessors (triple blinding). Bottles were labelled only with subject ID, and only the pharmacy and safety monitor had the key. Each bottle was equipped with a pump to dispense about 0.5 grams of the formulations described above as either a 1% rapamycin cream (High dose), a 0.1% rapamycin cream (Low dose), or a cream that contained only the vehicle and no rapamycin (Placebo).

Subjects applied the topical formula nightly over the affected areas, leaving the cream on overnight without washing. Patients were followed monthly for 6 months, for a total of seven visits (baseline=visit 1, after six months=visit 7). Photo-documentation occurred at baseline and monthly throughout the study.

In the initial portion of the clinical trial, blood levels of rapamycin were drawn to assess for absorption each month. During this time of the clinical trial no subject had any evidence of systemic rapamycin absorption. In fact, one subject (on low dose) lathered an entire month's therapy and had an undetectable rapamycin level the following day. Accordingly, the trial protocol was altered to discontinue serum measurements during the trial.

Baseline and final photographs were reviewed by two independent dermatologists masked both to treatment group and photo timing (baseline versus follow up).

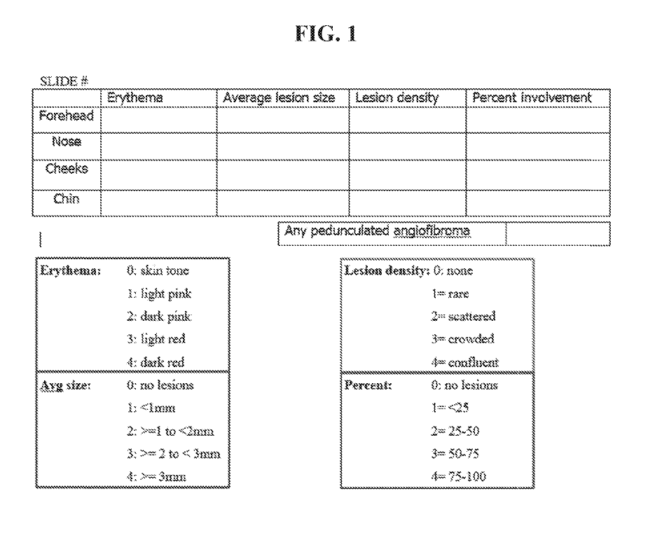

An Angiofibroma Rating Scale was also used to evaluate the presence, size, and density of angiofibromas found on individuals throughout the trial. Four Facial zones were evaluated (Forehead, Nose, Chin, Cheeks). There were 4 assessments made in each facial zone (Erythema, Size, Density, percent involvement). Each assessment had a 0-4 score. The score for each zone was calculated as (Erythema+Size+Density)×Percent Involvement. The Total angiofibroma scale (AGS)=sum of all 4 zones. Ten points were added to score for any pedunculated angiofibroma lesions. This gave a possible scoring range=0-202. FIG. 1 shows a diagram of a scoring chart used for calculating the AGS. In this evaluation, the Nose region extended to the nasofacial sulcus and the alar groove and skin immediately lateral to the nasal ala. The Chin region was defined by drawing a straight line down from the lateral commissures of the mouth.

The baseline demographics for the individuals participating in the trial are provided below in Table 1.

TABLE 1
Study Participants Demographics

| | Placebo | Low Dose | High Dose |
|---|---|---|---|
| | (n = 58) | (n = 61) | (n = 58) |
| Age (SD) | 22 (15) | 21 (12.4) | 19 (13.3) |
| Gender (% Male) | 29 (50%) | 31 (51%) | 23 (40%) |

| | Placebo | Low Dose | High Dose |
|---|---|---|---|
| | (n = 41) | (n = 45) | (n = 48) |
| Baseline AGS, | 39 (23-66) | 38 (26-68) | 37 (26-64.5) |
| Baseline AGS, | 43.5 (22.2) | 47.3 (27.3) | 45.5 (22.1) |
| mean | | | |

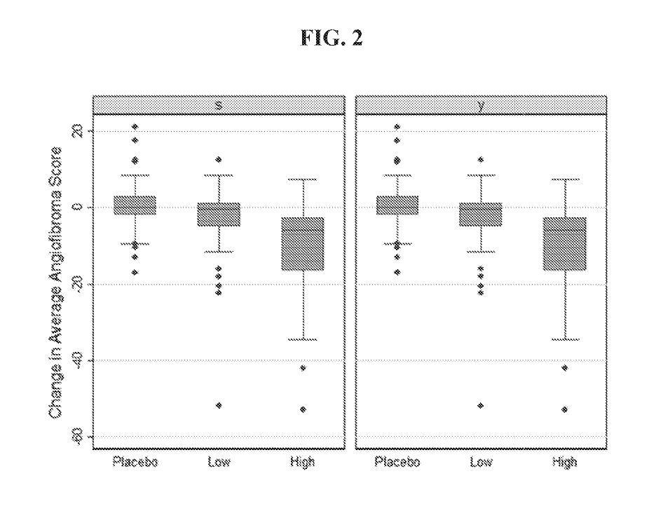

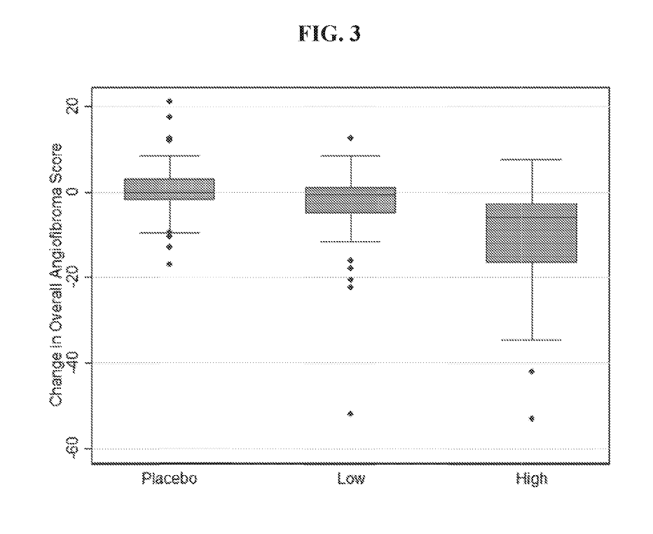

FIG. 2 shows the change in average AGS for the three treatment arms (placebo, low, and high) as reported for both dermatologists (indicated by shorthand as s and y). FIG. 3 shows the change in overall AGS for the three treatment arms (placebo, low, and high) combined for both dermatologists.

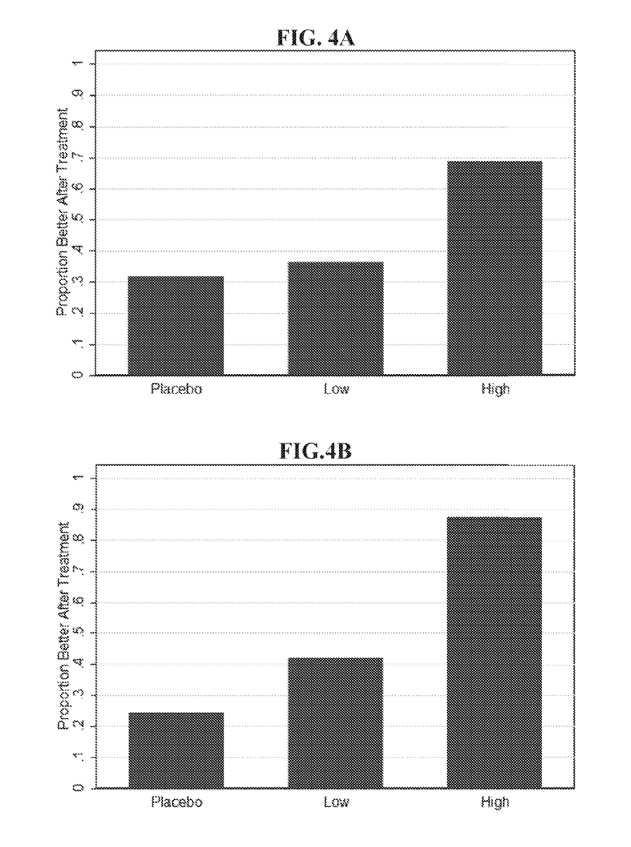

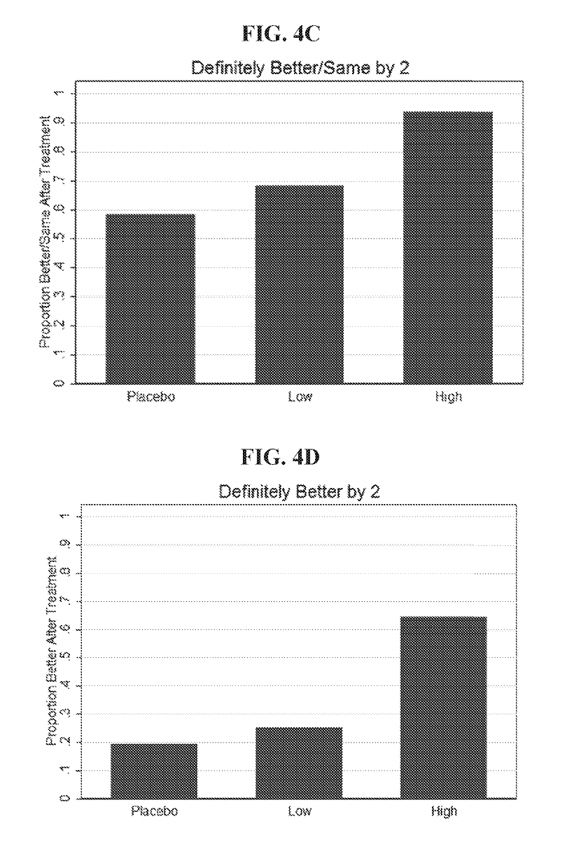

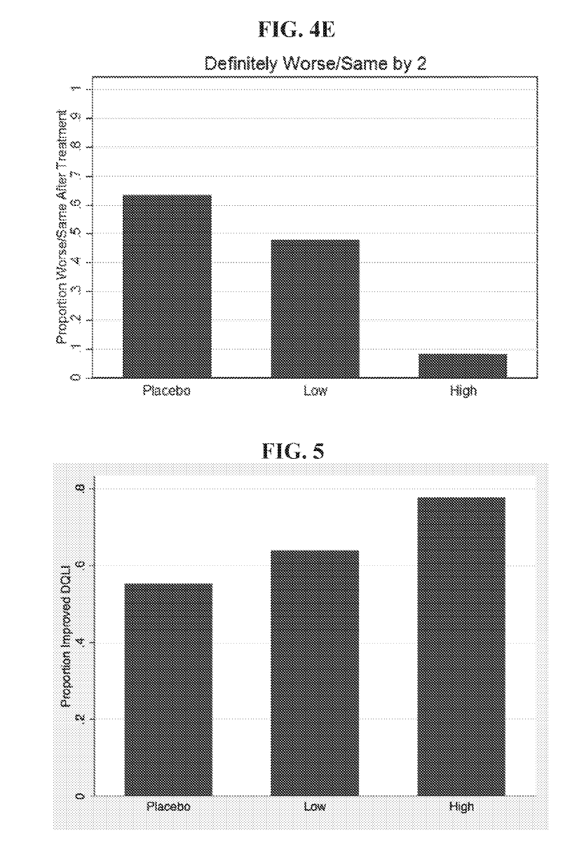

The dermatologists were also provided with a slide showing two photographs (A and B) of the same patient at baseline and after treatment and were asked to complete a form indicating in which photograph (A or B) the facial angiofibromas appeared to be less prominent. There was an option to select SAME if the lesions appeared the same in both photos. The dermatologists were allowed to zoom into the high resolution images of the patients as much as needed. FIG. 4A shows the proportion of patients in each treatment arm who were reported to have less prominent lesions after treatment compared to baseline, for the first dermatologist. FIG. 4B shows the proportion of patients in each treatment arm who were reported to have less prominent lesions after treatment compared to baseline, for the second dermatologist. FIG. 4C shows the proportion of patients in each treatment arm who were reported have less prominent or the same prominence of lesions after treatment compared to baseline for both dermatologists. FIG. 4D shows the proportion of patients in each treatment arm who were reported to have less prominent lesions after treatment compared to baseline for both dermatologists. FIG. 4E shows the proportion of patients in each treatment arm who were reported to have more prominent lesions after treatment compared to baseline for both dermatologists.

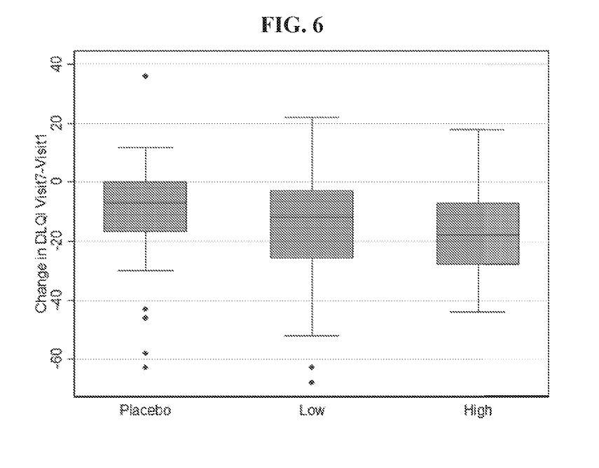

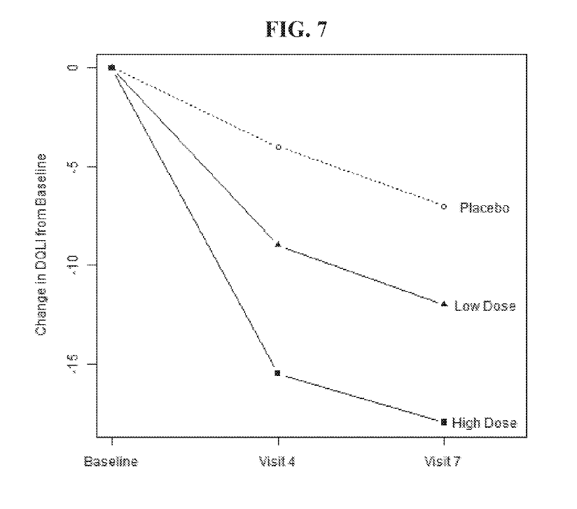

The patients were also asked about their quality of life during visits 1, 4, and 7. This was calculated using the Dermatology Quality of Life Index (DQLI), a commonly used questionnaire in cutaneous therapy clinical trials. The scores were summated and tabulated. FIG. 5 shows the proportion of patients in each treatment arm who reported improved DQLI between visits 1 and 7. FIG. 6 shows the change in DQLI for patients in each treatment arm between visits 7 and 1. FIG. 7 shows the change in DQLI from baseline for patients in each treatment arm for baseline and visits 4 and 7.

29 individuals (16%) did not complete the trial. Eighteen subjects discontinued the trial due to unspecified causes. Five subjects indicated non-compliance, 3 had an adverse event (AE), 2 started oral m-TOR inhibition, and one had an unrelated issue. Adverse events (AE), adverse events related to the trial (Related AEs) and related serious adverse events (Related SAEs) reported for all patients in the three treatment arms are shown below in Table 2. Table 3 shows all adverse events for each treatment arm sub-divided into the nature of the AE.

TABLE 2
Adverse Events

| | AEs | Related AEs | Related SAEs |
|---|---|---|---|
| Placebo (n = 58) | 13 (22%) | 8 (14%) | 0 of 2 |
| Low Dose (n = 61) | 20 (33%) | 10 (16%) | 0 of 2 |
| High Dose (n = 58) | 19 (33%) | 13 (22%) | 0 of 2 |

TABLE 3
Adverse Events Detail

| | Derm | Resp | GI | GU/GYN | Neuro | Other |
|---|---|---|---|---|---|---|
| Placebo | 8 | 3* | | | 1* | 1 |
| Low | 11 | 4 | 1* | 2 | 1 | 1* |
| High | 15 | 2 | 1* | | 1* | |

As seen in Tables 2 and 3, adverse events were common but usually minor. All related AEs were dermatologic in nature and none were related to the investigational product. Two serious AEs (SAEs) were reported in each treatment arm, and none were related to the investigation or investigational product. The SAEs included shortness of breath (SOB), subependymal giant cell astrocytomas (SEGA) resection, cholecystectomy, cellulitis, emesis, and hydrocephalus.

Overall, the results indicated that the topical rapamycin was well tolerated in most subjects. There were few treatment related dropouts or treatment ending AEs. The high dose topical rapamycin (1.0%) significantly improved all outcomes. The angiofibroma rating scale ratings showed a median change in average score for High (−6), Low (−0.5), and Placebo (0) (p<0.001). With regard to comparative improvement, a report of definitely better from both dermatologists was reported in all treatment arms, including High (65%) versus Low (25%) versus Placebo (20%) (p<0.001). Subject quality of life (p=0.037) and dose response trend (p=0.01) was also positive for the high dose investigational product.

What is claimed is:

1. A composition for use in treating facial angiofibromas, or other skin lesions, in a patient in need thereof, the composition comprising rapamycin in an amount of from 0.1% to 5% by weight, a liquid glycol, and a dermatologically acceptable carrier, wherein the composition is free of alcohol.

2. The composition of claim 1, wherein the patient in need of treatment is a patient diagnosed with Tuberous Sclerosis Complex (TSC).

3. The composition of claim 1, wherein the other skin lesions are selected from hemangiomas, vascular malformations, pyogenic granulomas, essential telangiectasias, familial multiple discoid fibromas, and cherry angiomas.

4. The composition of claim 3, wherein the vascular malformations are port wine stains or lymphangiomas.

5. The composition of claim 1, wherein the use is for treating other skin lesions and the patient in need of treatment is a patient diagnosed with Proteus, Brooke-Speigler syndrome, nevus sebaceous, epidermal nevus, oral lichen planus, chelitis granulomatosis, neurofibromatosis type 1, overgrowth syndromes, or gingival hypertrophy.

6. The composition of any one of claims 1-5, wherein the rapamycin is present in an amount of about 0.1% to about 1%, or from about 0.5% to 1%, or from about 1% to 3%, or from about 2.5% to 5%, or from about 3% to 5% by weight based on the total weight of the composition.

7. The composition of any one of claims 1-5, wherein the rapamycin is present in an amount of 1%, 2%, 3%, 4%, or 5% by weight based on the total weight of the composition.

8. The composition of any one of claims 1-7, wherein the composition is free of added formaldehyde, fragrances, and dyes.

9. The composition of any one of claims 1-7, wherein the composition is free of added acetone.

10. The composition of any one of claims 1-7, wherein the composition is free of added polyvinylidene fluoride.

11. The composition of any one of claims 1-10, wherein the liquid glycol is selected from propylene glycol, polyethylene glycol, and glycerol.

12. The composition of claim 11, wherein the liquid glycol is propylene glycol.

13. The composition of any one of claims 1-11, wherein the carrier comprises one or more of purified water, white petrolatum, sorbitol solution, cetearyl alcohol, propylene glycol, ceteareth-20, simethicone, glyceryl monostearate, polyethylene glycol monostearate, sorbic acid, and butylated hydroxytoluene (BHT).

14. A composition for use in treating facial angiofibromas in a patient in need thereof, the composition comprising rapamycin in an amount of from greater than 2% up to 5%, or from about 2.5% or 3% up to about 5% by weight, a liquid glycol in an amount of from 1-8% by weight, and a dermatologically acceptable carrier, wherein the composition is free of added alcohol, acetone, and polyvinylidene fluoride, and all weight percentages are based on the total weight of the composition.

15. The composition of claim 14, wherein the liquid glycol is selected from propylene glycol, polyethylene glycol, and glycerol.

16. The composition of claim 15, wherein the liquid glycol is propylene glycol.

17. The composition of any one of claims 1-16, wherein the composition is effective to treat facial angiofibromas as determined by a reduction in average size and number of lesions.

18. The composition of any one of claims 1-16, wherein the dermatologically acceptable carrier is formulated as a cream.
